(19) United States
(12) Patent Application Publication
(10) Pub. No.: US 2021/0142883 A1
(43) Pub. Date: May 13, 2021

(54) Integrated systems and methods for producing synergistic therapeutic and commercial effects

(71) Applicant: Jason O. Jaeger, D.C., P.C., Las Vegas, NV (US)

(72) Inventor: Jason Orion Jaeger, Las Vegas, NV (US)

(73) Assignee: Jason O. Jaeger, D.C., P.C., Las Vegas, NV (US)

(21) Appl. No.: 16/974,121

(22) Filed: Oct. 8, 2020

Related U.S. Application Data
(60) Provisional application No. 62/973,620, filed on Oct. 15, 2019.

(51) Int. Cl. G16H 20/40; G16H 20/60; G16H 20/30; G16H 30/40

(52) U.S. Cl. CPC G16H 20/40; G16H 30/40; G16H 20/30; G16H 20/60

(57) ABSTRACT

In one aspect, the present invention provides a system for providing therapeutic services to a client. The system comprises at least five different therapeutic services that complement one another. The therapeutic services are selected from a group of therapeutic services consisting of: a unified Medical, physical therapy and Chiropractic Biophysics™ approach, where the historically separate disciplines of science align structural and/or functional medical therapy, with, chiropractic biophysics spine rehabilitation, spinal adjustments, non-steroidal trigger point muscle or joint injections, dry needling, digitized ,measurable postural analysis using Posture Screen software, digital high-frequency full spine and sectional spine imaging, NCV/EMG, medically designed and physician-supervised, drug-free weight loss and body fat composition testing, 2-3 point bending spinal traction to correct scoliosis, loss of neck and low back curve, and reduction of thoracic hyperkyphosis, using a Clinical Biomechanics of Posture approach, scoliosis treatment using ScoliBrace and BraceScan, UTS Universal Tractioning Systems, and regenerative medicine using umbilical cord blood stem cells.

### Field Of The Invention

The present invention is generally related to integrated systems and methods for producing therapeutic and commercial effects. It is more specifically directed to systems and methods related to integrated medicine for producing therapeutic and commercial effects.

### BACKGROUND OF THE INVENTION

There have been reports of integrated medicine. US Pat. Appl. Pub. No. 2019/0221314, entitled “Integrated Precision Medicine by Combining Quantitative Imaging Techniques with Quantitative Genomics for Improved Decision Making”, for instance, allegedly discusses the following: “Disclosed herein is a combined multi-modality biomarker method for identification and treatment of a disease comprising performing quantitative and/or semi-quantitative molecular imaging on a patient; where the semi-quantitative imaging includes a cut-off; measuring a first plurality of parameters from the quantitative and/or semi-quantitative molecular imaging; simultaneously or sequentially performing a liquid biopsy on the patient; measuring a second plurality of quantitative and/or semi-quantitative molecular parameters from the liquid biopsy; developing an algorithm that combines one or more of the first plurality of parameters and one or more of the second plurality of parameters; where the algorithm is operative to identify a disease and/or predict a course of treatment and/or monitoring the patient; and treating the patient with the course of treatment generated by the algorithm.” Abstract.

US Pat. Appl. Pub. No. 2016/0235362, entitled “Integrated Medicine Method for Health Promotion” allegedly discusses the following: “An integrated medicine method for treating and preventing disease, and promoting health is established. The integrated medicine method utilizes western medicine, natural medicine, chinese medicine, functional medicine, and bio-energy/bio-information medicine to assess health and provide treatments and health adjustments. Disease treatment and prevention, and health promotion are provided, combining therapeutic, prophylactic, anti-aging and rejuvenile knowledge to establish an integrated medicine method. With the most advanced medical technology, ideas, products, pathways and networks, advanced types of health care are provided for all people's holistic health through comprehensive physical and mental health assessment using the methods of empirical science, health conditioning, maintenance, and health promotion.” Abstract.

Despite the various reports, there is still a need in the art for new systems and methods related to integrated medicine for producing therapeutic and commercial effects.

### SUMMARY OF THE INVENTION

In one aspect, the present invention provides a system for providing therapeutic services to a client. The system comprises at least five different therapeutic services that complement one another. The therapeutic services are selected from a group of therapeutic services consisting of: a unified Medical, physical therapy and Chiropractic Biophysics™ approach, where the historically separate disciplines of science align structural and/or functional medical therapy, with, chiropractic biophysics spine rehabilitation, spinal adjustments, non-steroidal trigger point muscle or joint injections, dry needling, digitized ,measurable postural analysis using Posture Screen software, digital high-frequency full spine and sectional spine imaging, NCV/EMG, medically designed and physician-supervised, drug-free weight loss and body fat composition testing, 2-3 point bending spinal traction to correct scoliosis, loss of neck and low back curve, and reduction of thoracic hyperkyphosis, using a Clinical Biomechanics of Posture approach, scoliosis treatment using ScoliBrace and BraceScan, UTS Universal Tractioning Systems, and regenerative medicine using umbilical cord blood stem cells.

In another aspect, the present invention provides a method for providing therapeutic treatment to a patient. The method comprises: performing an observational analysis regarding the patient; providing a first treatment to the patient; collecting data regarding the first treatment and performing a first data analysis, wherein the data collection and first data analysis comprise the use of at least one data collection and analysis tool, and wherein the data and collection analysis tool is selected from a group consisting of digital high-frequency full spine and sectional spine imaging, electromyography, nerve conduction velocity, and transcranial magnetic stimulation; either continuing the first treatment or providing a second treatment to the patient based on the collection and analysis of data related to the first treatment; collecting data regarding the continuing first treatment or second treatment and performing a second data analysis, wherein the data collection regarding the continuing first treatment or second treatment and second data analysis comprise the use of at least one data collection and analysis tool, and wherein the data and collection analysis tool is selected from a group consisting of digital high-frequency full spine and sectional spine imaging, electromyography, nerve conduction velocity, and transcranial magnetic stimulation; repeating either the first treatment, the second treatment or both on the patient; thereby providing therapeutic treatment to the patient.

In another aspect, the present invention provides a method for increasing revenue for a healthcare professional. The method comprises: performing a first therapeutic service on a patient; collecting data regarding the first therapeutic service and performing a first data analysis, wherein the data collection and first data analysis comprise the use of at least one data collection and analysis tool, and wherein the data and collection analysis tool is selected from a group consisting of digital i high-frequency full spine and sectional spine imaging, electromyography, nerve conduction velocity, and transcranial magnetic stimulation; either modifying the first therapeutic service or performing a second therapeutic service on the patient; if modifying the first therapeutic service, collecting data regarding the modified therapeutic service and performing a second data analysis, wherein the data collection and first data analysis comprise the use of at least one data collection and analysis tool, and wherein the data and collection analysis tool is selected from a group consisting of digital Fuji high-frequency full spine and sectional x-rays, electromyography, nerve conduction velocity, and transcranial magnetic stimulation; if providing the second therapeutic service, collecting data regarding the second therapeutic service and performing a third data analysis, wherein the data collection and first data analysis comprise the use of at least one data collection and analysis tool, and wherein the data and collection analysis tool is selected from a group consisting of digital Fuji high-frequency full spine and sectional x-rays, electromyography, nerve conduction velocity, and transcranial magnetic stimulation; analyzing and comparing data from the first, second and third data analysis; providing further therapeutic services to the patient; thereby increasing revenue for the healthcare professional.

### BRIEF DESCRIPTION OF THE DRAWINGS

FIG. 1 shows an embodiment of a system according to the present invention.

FIG. 2 shows an embodiment of a therapeutic method according to the present invention.

FIG. 3 shows an embodiment of a commercial method according to the present invention.

### DETAILED DESCRIPTION OF THE INVENTION

“Physiotherapy” refers to the treatment of injury, disease and disorders through physical methods, such as exercise, posture retraining 2-3 point bending corrective traction, massage, manipulation and other treatments. There are different areas of physiotherapy, including the following: musculoskeletal (orthopedic physiotherapy), used to treat conditions such as sprains, back pain, arthritis, strains, incontinence, bursitis, posture problems and reduced mobility; neurological, used to treat disorders of the nervous system, including strokes, spinal cord injuries, acquired brain injuries, multiple sclerosis and Parkinson's disease; cardiothoracic, used to treat asthma, chronic bronchitis, emphysema and other cardio-respiratory disorders. Therapies used by physiotherapists include, without limitation, the following: joint manipulation and mobilization; manual resistance training; stretching; muscle strengthening; posture re-training; cardiovascular stretching and training; electrotherapy techniques, such as Transcutaneous Electrical Nerve Stimulation (“TENS”), laser therapy, diathermy and ultrasound; electrotherapies, such as functional electrical stimulation (“FES”), electromyography feedback and transcranial magnetic stimulation (“TMS”).

“Chiropractic adjustment” refers to a procedure in which a trained specialist (chiropractic physician) uses his hands or a small instrument to apply a controlled, sudden force to a spinal joint.

“Trigger point injection” (“TPI”) refers to a procedure used to treat painful areas of muscle that contain trigger points, or knots of muscle, that form when muscles do not relax. In a TPI procedure, a healthcare professional inserts a small needle for injection into a patient's trigger point. The injection typically contains a local anesthetic or saline (non-steroidal TPI); in steroidal TPIs the injection includes a corticosteroid. TPI may be used to treat several different muscle groups, such as those in the arms, legs, lower back and neck. It can also be used to treat fibromyalgia, tension headaches and myofascial pain syndrome.

“Dry needling” refers to an intervention that uses a thin filiform needle to penetrate the skin and stimulate underlying myofascial trigger points, muscular, and connective tissues for the management of neuromusculoskeletal pain and movement impairments.

“Postural analysis” refers to an analysis used to determine which areas of a patient's body are under more strain than others and which muscle groups are causing this strain.

“Posture Screen software” refers to a movement analysis software designed for chiropractors who wish to screen for movement and postural dysfunction. The software is intended to be an objective postural and movement screening tool allowing findings to be quickly communicated to patients/prospective patients. The software also has a body composition analysis module that provides for assessment of a person's percent body fat (PBF), body mass index (BMI), basal metabolic rate (BMR), waist hip ratio (WHR), lean body mass (LBM) and fat body mass (FBM).

“Nerve Conduction Velocity” (“NCV”) refers to a test that determines how fast electrical signals move through a nerve. The test is done along with electromyography (“EMG”) to assess the muscles for abnormalities.

“Electromyography” (“EMG”) refers to a test that measures how well a person's muscles respond to nerve signals from the brain.

“2-3 Point Bending Corrective Traction” refers to the use of weights and/or other forces to treat skeletal and muscular disorders, pinched or compressed nerves, and injuries to joints, vertebrae, muscles and ligaments by stretching the muscles and ligaments and increasing the space between vertebrae.

“Clinical Biomechanics of Posture” (“CBP”) refers to patient rehabilitation that uses rotations and translations to describe the spine and posture parts during both displacement analysis and correction. Rooted in Linear Algebra, CBP used applied forces and human body positioning to restore normal posture and normal spinal alignment. Original CBP approaches, by Harrison, include Mirror Image postural adjusting with a drop table and/or an instrument, Mirror Image postural exercises, Mirror Image postural traction and sagittal curve traction.

“Scoliosis” refers to a sideways curvature of the spine that occurs most often during the growth spurt just before puberty. While scoliosis can be caused by conditions such as cerebral palsy and muscular dystrophy, the cause of most scoliosis is unknown.

“Scolibrace” refers to an over-corrective brace. It works by guiding the body and spine into a posture that is the opposite of how the scoliosis is shaped.

“BraceScan” refers to a system that combines 3D full-body laser scanning technology, x-rays and posture photographs. It provides for the custom design of Scolibraces using Computer Aided design (CAD) and then Computer Aided Manufacture (CAM).

“Regenerative medicine” refers to the process of replacing or “regenerating” human cells, tissues or organs to restore or establish normal function.

“Stem Cell” refers to an undifferentiated cell of a multicellular organism which is capable of giving rise to indefinitely more cells of the same type, and from which certain other kinds of cell arise by differentiation.

“Haematopoietic (blood) stem cells” (“HSC”s) are contained in the umbilical cord and placenta of a baby after birth. HSCs can make every type of cell in the blood—red cells, white cells and platelets.

“Transcutaneous electrical nerve stimulation” refers to therapy that involves the use of low-voltage electric currents to treat pain. Electrodes or mediums for electricity to travel to the body, placed on the body at the site of pain deliver electricity that travels through the nerve fibers.

“Functional electrical stimulation” refers to a technique that uses low-energy electrical pulses to artificially generate body movements in individuals who have been paralyzed due to injury to the central nervous system.

“Transcranial magnetic stimulation” refers to a noninvasive procedure that uses magnetic fields to stimulate nerve cells in the brain.

The present invention is generally related to integrated systems and methods for producing therapeutic and commercial effects. It is more specifically directed to systems and methods related to integrated medicine for producing therapeutic and commercial effects.

FIG. 1 shows an embodiment of a system (100) according to the present invention. As shown, there are ten different components of the system: structural and/or functional physiotherapy (102); chiropractic spinal adjustments (104); non-steroidal trigger point muscle or joint injections (106); postural analysis using Posture Screen software (108); digital Fuji high-frequency full spine and sectional x-rays (110); NCV/EMG (112); medically designed and physician-supervised, drug-free weight loss and body fat composition testing (114); Traction—Clinical Biomechanics of Posture (116); Scoliosis treatment using ScoliBrace and BraceScan (118); Regenerative Medicine using umbilical cord blood stem cells (HSCs, 120). It should be understood that other embodiments of the system may include less components (e.g., 102, 104, 106, 108 and 110) or more.

Systems of the present invention provide the basis for both synergistic therapeutic and commercial effects. FIG. 2 shows an embodiment of a therapeutic method (200) according to the present invention. The method starts at 202. An observational analysis regarding a patient is made by a healthcare professional at 204. Nonlimiting examples of such analyses include the following observations: a sprain, back pain, arthritis, a strain, incontinence, bursitis, posture problems, reduced mobility, a stroke, a spinal cord injury, an acquired brain injury, multiple sclerosis, Parkinson's disease, asthma, chronic bronchitis, emphysema, cardiovascular disorders, fibromyalgia, tension headaches, myofascial pain syndrome, skeletal and muscular disorders, pinched or compressed nerves, joint injuries, scoliosis.

At 206, Treatment 1 is provided to the patient. Nonlimiting examples of such a treatment include: exercise guidelines, massage, manipulation, joint manipulation and mobilization, manual resistance training, stretching, muscle strengthening, posture re-training, cardiovascular stretching and training, electrotherapy, trigger point injection, traction, stem cell injection. A first Data Collection and Analysis is performed at 208. Nonlimiting examples of data collection and analysis tools include: digital Fuji high-frequency full spine and sectional x-rays; Electromyography; Nerve Conduction Velocity; and, Transcranial Magnetic Stimulation. The first data collection and analysis informs the healthcare professional as to whether Treatment 1 should be continued (210), whether alternative Treatment 2 (212) should be performed, or whether both Treatment 1 and Treatment 2 should be performed. The Treatment 2 can be one of those listed as treatments relative to Treatment 1, with the understanding that Treatment 1 and Treatment 2 are different members of the list.

At 214, a second Data Collection and Analysis is performed (e.g., digital Fuji high-frequency full spine and sectional x-rays; Electromyography; Nerve Conduction Velocity; or, Transcranial Magnetic Stimulation). The second data collection and analysis informs the healthcare professional whether Treatment 1 (210), Treatment 2 (212), or both, should be repeated or whether the method is ended at 216.

One benefit of method 200 is that the data collection and analysis can be used to modify or change treatment, resulting in an enhanced therapeutic outcome. Nonlimiting examples of outcome include: pain reduction; reduction of arthritis symptoms; enhanced or corrected posture; reduction of number of headaches; alleviation of pinched or compressed nerves; enhanced breathing; enhanced mobility; reduction in scoliosis. In certain cases, the method provides for at least a 5% beneficial difference in outcome over the use of Treatment 1 in the absence of the Data Collection and Analysis steps (e.g., additional pain reduction of at least 5% as measured by a suitable pain scale). Nonlimiting examples of pain scales include: Numerical Rating Pain Scale; Wong-Baker Faces Pain Scale; FLACC Scale; CRIES Scale; COMFORT Scale; and, McGill Pain Scale. In other cases, the method provides for at least a 10%, at least a 15%, at least a 20%, or at least a 25% beneficial difference in outcome.

FIG. 3 shows an embodiment of a commercial method (300) according to the present invention. The method starts at 302. At 304, a first therapeutic service is performed by a healthcare professional on a patient. Nonlimiting examples of such a therapeutic service include: exercise guidelines, massage, manipulation, joint manipulation and mobilization, manual resistance training, stretching, muscle strengthening, posture re-training, cardiovascular stretching and training, electrotherapy, trigger point injection, traction, stem cell injection. A first Data Collection and Analysis is performed at 306. Nonlimiting examples of data collection and analysis tools include: digital Fuji high-frequency full spine and sectional x-rays; Electromyography; Nerve Conduction Velocity; and, Transcranial Magnetic Stimulation.

The first Data Collection and Analysis informs the healthcare professional whether Service 1 should be modified (308), whether Service 2 (310, same list as Service 1 but different than Service 1) should be performed, or both. If step 308 is performed, then a second Data Collection and Analysis (312) is conducted; if step 310 is performed, then a third Data Collection and Analysis (314) is conducted. For either 312 or 314, suitable data collection and analysis tools—such as digital Fuji high-frequency full spine and sectional x-rays, Electromyography, Nerve Conduction Velocity, and, Transcranial Magnetic Stimulation—are used. Analyzed data from 312 and/or 314 are compared and analyzed at 316. Analysis 316 informs the healthcare professional whether further services should be provided to the patient. The method ends at 320.

One benefit of method 300 is that the data collection and analysis can be used to support treatment modification or the provision of other treatments. This increases the yearly per patient revenue of the healthcare professional. In certain cases, the yearly per patient revenue of a healthcare professional is increased at least 5% over the yearly per patient revenue in the absence of the Data Collection and Analysis. In other cases, the method provides for at least a 10%, at least a 15%, at least a 20%, or at least a 25% increase in yearly per patient revenue.

1. A system for providing therapeutic services to a client, wherein the system comprises at least five different therapeutic services that complement one another, and wherein the therapeutic services are selected from a group of therapeutic services consisting of: structural and/or functional physiotherapy, chiropractic spinal adjustments, non-steroidal trigger point muscle or joint injections, postural analysis using Posture Screen software, digital high-frequency full spine and sectional spinal imaging, NCV/EMG, medically designed and physician-supervised, drug-free weight loss and body fat composition testing, traction using Clinical Biomechanics of Posture, scoliosis treatment using ScoliBrace and BraceScan, and regenerative medicine using umbilical cord blood stem cells.

2. A method for providing therapeutic treatment to a patient, wherein the method comprises:
performing an observational analysis regarding the patient;
providing a first treatment to the patient;
collecting data regarding the first treatment and performing a first data analysis, wherein the data collection and first data analysis comprise the use of at least one data collection and analysis tool, and wherein the data and collection analysis tool is selected from a group consisting of digital Fuji high-frequency full spine and sectional x-rays, electromyography, nerve conduction velocity, and transcranial magnetic stimulation;
either continuing the first treatment or providing a second treatment to the patient based on the collection and analysis of data related to the first treatment;
collecting data regarding the continuing first treatment or second treatment and performing a second data analysis, wherein the data collection regarding the continuing first treatment or second treatment and second data analysis comprise the use of at least one data collection and analysis tool, and wherein the data and collection analysis tool is selected from a group consisting of digital high-frequency full spine and sectional spinal imaging, electromyography, nerve conduction velocity, and transcranial magnetic stimulation;
repeating either the first treatment, the second treatment or both on the patient; thereby providing therapeutic treatment to the patient.

3. A method of increasing revenue for a healthcare professional, wherein the method comprises:
performing a first therapeutic service on a patient;
collecting data regarding the first therapeutic service and performing a first data analysis, wherein the data collection and first data analysis comprise the use of at least one data collection and analysis tool, and wherein the data and collection analysis tool is selected from a group consisting of digital high-frequency full spine and sectional spinal imaging, electromyography, nerve conduction velocity, and transcranial magnetic stimulation;
either modifying the first therapeutic service or performing a second therapeutic service on the patient;
if modifying the first therapeutic service, collecting data regarding the modified therapeutic service and performing a second data analysis, wherein the data collection and first data analysis comprise the use of at least one data collection and analysis tool, and wherein the data and collection analysis tool is selected from a group consisting of digital high-frequency full spine and sectional spinal imaging, electromyography, nerve conduction velocity, and transcranial magnetic stimulation;
if providing the second therapeutic service, collecting data regarding the second therapeutic service and performing a third data analysis, wherein the data collection and first data analysis comprise the use of at least one data collection and analysis tool, and wherein the data and collection analysis tool is selected from a group consisting of digital high-frequency full spine and sectional spinal imaging , electromyography, nerve conduction velocity, and transcranial magnetic stimulation;
analyzing and comparing data from the first, second and third data analysis;
providing further therapeutic services to the patient
thereby increasing revenue for the healthcare professional.
